(19) United States
(12) Patent Application Publication
(10) Pub. No.: US 2009/0297631 A1
(43) Pub. Date: Dec. 3, 2009

(54) SALT COMPOSITIONS AND METHODS OF MAKING THE SAME

(75) Inventors: Jessica Adkins, Kimball Township, MI (US); Martin N. Aschauer, Port Huron, MI (US)

Correspondence Address: CARGILL, INCORPORATED, P.O. Box 5624, MINNEAPOLIS, MN, 55440-5624, US

(73) Assignee: CARGILL, INCORPORATED, Wayzata, MN (US)

(21) Appl. No.: 12/475,233

(22) Filed: May 29, 2009

Related U.S. Application Data
(60) Provisional application No. 61/130,360, filed on May 30, 2008.

(51) Int. Cl. A61K 33/14; A23G 3/02; A23L 1/221; A23L 1/30

(52) U.S. Cl. 424/680; 426/512; 426/650; 426/72

(57) ABSTRACT

Disclosed is a method of preparing a salt composition comprising mixing a salt and an ingredient, pressing the salt and ingredient together under pressure to form an agglomerated salt and ingredient composition in the form of a sheet, briquette, pellet, flake or other form, and reducing the salt and ingredient composition into particulates having a desirable size, where the particulates form a substantially uniform blended product of the salt and the ingredient. In one embodiment, the salt composition may be sodium chloride or a sea salt and a seasoning or flavoring agent.

### CROSS REFERENCE TO RELATED APPLICATION

This application claims the benefit of the U.S. Provisional Patent Application Ser. No. 61/130,360, filed 30 May 2008, entitled PREPARATION OF MULTI-COMPONENT SOLID FORMULATIONS WITH UNIFORM COMPOSITION IN EACH PARTICLE, which is hereby incorporated by reference in its entirety.

### BACKGROUND

Salt compositions of multiple components are usually in the form of simple blends or mixtures of ingredients. Existing methods for mixing and preparing such compositions result in mixtures wherein the various ingredients that make up the mixture have a propensity to partially separate during handling and storage. As such, the ingredients and components are not uniformly distributed throughout such compositions. These non-uniformly blended products can cause localized low or high concentrations of ingredients during their application on food products. For example, a non-uniformly blended salt and spice mixture may result in food tasting salty on one portion, while leaving a strong spicy flavor on another portion. Thus, traditional non-uniformly blended salt compositions or mixtures can be both frustrating to a user seeking a desired taste, and ineffective due to a non-uniform and uneven distribution of ingredients.

### SUMMARY

In an embodiment of the present invention, a method of preparing a salt composition comprises mixing a salt and an ingredient, pressing the salt and ingredient together under sufficient pressure to form an agglomerated composition, and comminuting the size of the agglomerated composition to a composition where the composition comprises particulates and a particulate comprises a substantially uniform blend of the salt and the ingredient. The composition can be used on or in a variety of food products, a dietary product, or a pharmaceutical product. The salt and ingredient can be pressed together by compacting them into an agglomerated composition that is, for instance, a continuous sheet, a briquette, or flakes, and subsequently comminuted or reduced into particulates having a desired size. An average particulate is a composition of the salt and the ingredient, and together the particulates form a substantially uniform composition of the salt and ingredient. A typical particulate of the composition will impart a similar taste profile, aroma and color to each portion of the food product to which it is applied. The particulates can be further separated between conforming and nonconforming sizes via a separation device. At least a portion of the nonconforming composition can be recycled back into the process in the mixing, pressing or comminuting steps.

In other embodiments, a salt composition is prepared wherein the composition comprises a salt such as sodium chloride and a flavoring agent, seasoning agent, coloring agent, aroma agent, masking agent, enhancing agent, high potency agent, compounded flavor, extract, spice, nutrient, micronutrient, spice, oil, herb, mineral, inorganic salt, organic salt, salt substitute, sweetener, vitamin, emulsifying agent, stabilizing agent, anti-caking agent, dietary supplement, antioxidant, or combinations thereof. In another embodiment the composition may include a sodium chloride salt or sea salt and a flavoring or seasoning agent. The composition may exhibit altered or enhanced physical properties such as, for example, a high solubility rate, low bulk density, and/or improved adherence to a product or substrate.

These and other aspects of the present invention are elucidated further in the detailed description.

### BRIEF DESCRIPTION OF THE DRAWINGS

The present invention will become more fully understood from the detailed description given below and the accompanying drawings. These drawings are given by way of illustration only, and thus are not intended to be limiting of the present invention.

FIG. 1 is a block diagram that illustrates an example of a process for preparing a composition of a salt and an ingredient by the steps of mixing, pressing, reducing (or commimuting).

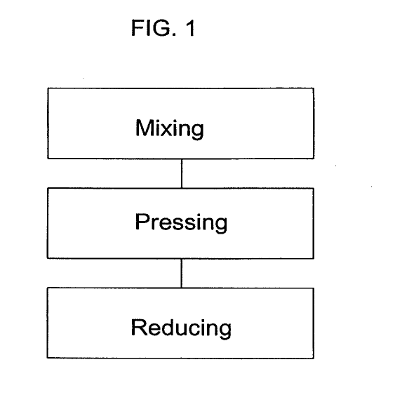

FIG. 2 is a block diagram that illustrates another example of a process for preparing a composition of a salt and an ingredient by the steps of mixing, pressing, and reducing (or commimuting) and separating, where the separated nonconforming product can be recycled back into any of the previous steps.

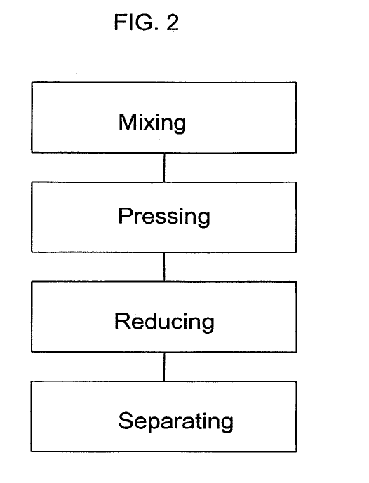

FIG. 3 is a block diagram that illustrates another example of a process for preparing a composition of a salt and an ingredient by the steps of mixing, pressing, separating, and reducing (or commimuting) and separating, where the separated nonconforming product can be recycled back into any of the previous steps.

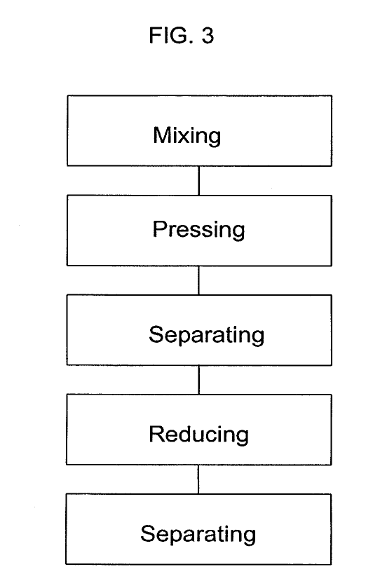

FIG. 4 is a schematic diagram that illustrates an example of a process for preparing a composition of a salt and an ingredient by the steps of mixing, pressing, and reducing (or commimuting) and separating, where the separated nonconforming product can be recycled back into any of the previous steps.

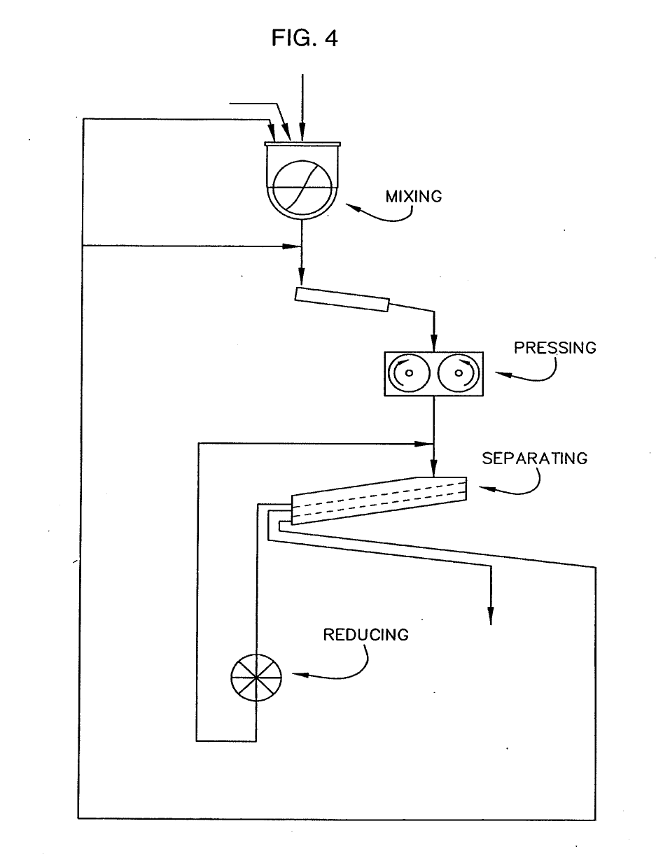

### DETAILED DESCRIPTION

The following description of the invention is intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

In an embodiment of the present invention, a method is described for preparing a composition comprising mixing a salt with an ingredient. A variety of salts or combinations of salts suitable for human consumption can be used in forming the salt compositions. For example, a salt can include a free flowing inorganic edible salt that is suitable for consumption, a monovalent or polyvalent salt of a polybasic food acid, or an amino acid or salt thereof. Another example of a salt can include an alkali metal or alkaline earth metal component such as sodium, potassium, magnesium, calcium, or similar ions either individually or in combination, present as the chlorides, sulfates, acetates, carbonates, gluconates or combinations thereof. Other types of salts include a chloride salt, such as sodium chloride, sea salt, potassium chloride, Fleur de Sel (or Flor de Sal), iodized salt, rock salt, or mixtures thereof. A salt can include trace amounts of other compounds, such as the trace minerals naturally present in sea salt or Fleur de Sel, or the trace amounts of iodine present in many edible salts such as iodized salt where the iodine is present in the form of sodium iodide, potassium iodide, or potassium iodate.

Examples of an ingredient that may be combined with a salt in any of the embodiments described herein include, but are not limited to, flavoring agents, masking agents, seasoning agents, coloring agents, aroma agents, enhancing agents, high potency agents, compounded flavors, extracts, nutrients, micronutrients, spices, herbs, oils, minerals, organic salts, inorganic salts, salt substitutes, sweeteners, vitamins, emulsifying agents, stabilizing agents, anti-caking agents, antioxidants, dietary supplements, or combinations thereof. By way of example and not limitation, an ingredient can also include any food ingredient that is or becomes recognized as generally recognized as safe (“GRAS”) such as those included in Title 21 of the Code of Federal Regulations (“CFR”) Parts 182, 184, and 186, in the Flavor and Extract Manufacturer's Association (“FEMA”) list, and other similar food ingredient lists in other countries. In certain embodiments, the salt substitutes may include, for example, potassium chloride, any salt substitute suitable for human consumption, or any combinations thereof. In certain embodiments, the masking agents or sweeteners may include, for example, sugar, any ingredient suitable for masking the bitterness or off-flavors of other compounds and suitable for human consumption, or any combination thereof. For instance, potassium chloride, a common salt substitute, is often used in conjunction with an ingredient that can mask the bitter or metallic taste often associated with it. Examples of masking agents or sweeteners that can be used include compounds such as autolyzed yeast extracts, amino acid derivatives, sucralose, stevia, simple sugars, monosodium glutamate, or combinations thereof. In certain embodiments, the flavoring agent is a chemical compound or composition suitable for human consumption found to have utility in the alteration of flavor or flavor characteristics of a substance, whether naturally occurring or synthetic. By way of example flavoring agents may include, but are not limited to, garlic, onion, mesquite, hickory smoke, butter, barbeque, or any combination thereof. In certain embodiments, the seasoning agents may include any spice, herb or seasoning such as Cajun, pepper, lemon pepper, soy, cheese, chili, ethnic seasoning combinations such as Mexican, That, Indian, or Italian, any seasoning suitable for human consumption, or any combination thereof. Examples of high potency agents can include compounds such as high intensity sweeteners, flavor enhancers such as monosodium glutamate, or modified food starch (for texture). In certain embodiments, the coloring agents may include, for example, any natural or artificial coloring agents suitable for human consumption, or any combination thereof. The above ingredients may be used alone or in various combinations to form the salt composition.

The salt and ingredient are physically combined by first mixing them together, pressing the mixture together under sufficient pressure to form an agglomerated composition, and comminuting the size of the agglomerated salt and ingredient composition into a composition where the composition comprises particulates, and an average particulate is a substantially uniform blend of the salt and ingredient. Composition and particulates can be further separated into groups, depending on the composition size desired, between nonconforming and conforming size. Conforming size can be the target size desired, whereas the nonconforming size may be oversized or undersized product in relation to the conforming size. Nonconforming particulates and composition can be recycled back into the process at a variety of locations, such as in the mixing, pressing or comminuting steps for further mixing, pressing or comminuting. Because the particulates and the composition comprise a substantially uniform blend of the salt and the ingredient, they can be recycled back into the process to obtain a conforming size. In contrast, product blended by known methods will have separation issues from handling resulting in a non-uniform blend of the salt and ingredient.

Mixing methods to mix the salt and ingredient can include placing the salt and ingredient into a mixing device. The salt and ingredient can be placed into the mixing device by means commonly known in the industry, such as by hand or by a conveyor. The mixing device can be, for example, a ribbon blender, paddle blender, plow blender, twin shell blender, double-cone blender, V-blender, single or double rotor blender, müller blender, vertical screw mixer, or similar mixing systems. The salt and ingredient can be placed into the mixing device for a sufficient amount of time to thoroughly mix the salt and ingredient. The mixing can be done either by batch mixing or a continuous process. In one batch embodiment the salt and ingredient are mixed in the mixing device for about two to twenty minutes. In another embodiment the salt and ingredient are mixed in the mixing device for about four to eight minutes.

Pressing methods to form the salt and ingredient into an agglomerated composition can include compacting, flaking, briquetting, or other similar techniques. Once the salt and ingredient are mixed in the mixing device, they can be conveyed to the pressing device. For compacting or briquetting with a device such as a compactor or briquettor made by Bepex International LLC, the mixture can be force fed into the nip angle of the pressing device, such as a dual roll press with counter-rotating rolls. When the press method is flaking, such as that done with the Ludman Machine Company flaking mill, generally the mixture is gravity-fed into the press and metered or regulated to ensure a uniform distribution of the particles. This allows a thin uniform distribution of the salt and ingredient mixture to be introduced into the press much like a “waterfall” of the mixture.

Pressing can be performed by compacting the salt and ingredient together under pressure until the salt and ingredient are formed into an agglomerated composition such as, for example, a sheet, pellet, briquette, or other form suitable for manufacturing and/or handling. The pressing device can include, for example, a dual or twin roll press with counter rotating rolls, dual roll briquette press, dual roll compactor, flaking roll, and other similar pressing devices. In certain embodiments, the salt and ingredient are compacted into one or more thin sheets or flakes or pressed into briquettes. In a preferred embodiment, the sheet is a continuous sheet having an average thickness of at least 0.005 inches. In another embodiment the sheet is a continuous sheet having an average thickness of about 0.005 inches to about 0.3 inches. In a preferred embodiment, the salt and ingredient are pressed together to form an agglomerated composition of flakes where the flakes have an average thickness of at least 0.004 inches. In another embodiment, the flakes have an average thickness of about 0.004 inches to about 0.05 inches. In a preferred embodiment, the briquette has an average thickness of at least 0.25 inches. In another preferred embodiment, the briquette has an average thickness of about 0.25 inches to about 2.0 inches.

In one embodiment, the pressing step is run at a roll pressure of between about 0.33 and 1.33 tons per lineal inch, at a roll speed of between about 0.5 and 25 feet per second (fps). In another embodiment, the roll speed is between about 2.5 and 15 fps. In another embodiment, the pressing step is run at a roll pressure of about 1.0 tons per lineal inch at a roll speed of about 6.25 fps at ambient temperature. Each particulate or particle formed by the compaction process is a substantially uniform blend of the salt and ingredient, and a multitude of such particulates make up a sheet, flake, briquette, pellet or other form. Pressing the salt and ingredient together under pressure helps prevent separation of the salt and ingredient e.g., during handling, storage and/or transportation and forms a uniformly blended particulate and composition. Accordingly, the particles will impart a similar taste, color and aroma to each portion of a substrate to which the composition is applied and will exhibit other benefits such as uniform visual appearance, greater adherence, lower bulk density and faster solubility.

The agglomerated composition can then be comminuted, for example, by means of a roller mill, disk mill, pin mill, jaw crusher, hammer mill, jet mill, ball mill, and other similar machines. Communitng the size of the agglomerated composition in the form of a sheet, flakes, pellets or briquettes allows the product to be a composition of the particulates in a desired size for use in or on a food product or substrate. The resulting particulates will form a composition that is a substantially uniform blend of the salt and ingredient. The size of the particle can depend on the end-use of the final product. By way of example, a smaller particle size may be desired for end products such as chips. Other applications, such as on pretzels, may require a larger particulate. In a preferred embodiment, particulate sizes may range from about 0.005 inches to 2.0 inches.

The particulates can be further separated according to size via a separation device. For instance, particulates can be further separated into groups of certain size particulates depending on the product or substrate with which it will be used, the form in which it will be sold, or the type of starting material. For example, if particulates of a target (conforming) size are desired, particulates that are oversized or undersized (nonconforming) as compared to the target size can be further separated out from the conforming sized particulates and composition. The nonconforming particulates and composition can be recycled back into the process at a variety of locations such as in the mixing, pressing, or reducing steps for further mixing, pressing or comminuting. In a preferred embodiment, the amount of nonconforming material to be recycled back into the process can be in the range of 0% to 100% of the material. In other words, at least a portion of the nonconforming composition (or particulates) can be recycled back into the process in the mixing, pressing or comminuting steps. Examples of separation devices can include a sieving device, sieve shaker, vibratory screen and other similar separation devices.

The particulates or particles formed by the above method into the composition will exhibit altered or enhanced physical properties such as, for example, a high solubility rate, low bulk density, and/or improved adherence. For example, a composition in the present invention that includes sodium chloride as the salt will have a solubility rate faster than a corresponding product prepared by blending the salt and ingredient having the same average particle weight. In addition, it will have an apparent bulk density that is less than a corresponding product prepared by blending the salt and ingredient having the same average particle weight. Moreover, the composition will have an improved adherence to food than a corresponding product prepared by blending the salt and ingredient having the same average particle weight. The particulates and composition may exhibit one or more of these altered or enhanced physical properties.

The agglomerated salt and ingredient composition, whether as a sheet, flake, pellet, briquette, or any other form, may be subsequently comminuted to a preferred particle size suitable for distribution. For example, a sheet may be subsequently comminuted to one or more smaller particles or particulates, where each particle or particulate is a composition of the salt and ingredient. In certain embodiments, a sheet or other solid form may be reduced to a size suitable for insertion in a salt grinder that may then be used to create smaller particles for consumer use. A sheet or other solid form may be reduced to one or more particles having a size suitable for application to various food substrates, for example, as toppings for a pretzel or snack chip.

The composition of the present invention may have any variety of applications, including with a product such as a food product, a dietary product, or a pharmaceutical product. In one embodiment, the composition can be used in or on a food product such as a meat, grain, vegetable, fruit, sauce, prepared meal, frozen food, candy, snack, chip, pre-packaged product, beverage, or combination thereof. In another embodiment the composition can be used in or on a dietary product such as a dietary supplement, energy bar, beverage, powder, additive, energy drink, retort, aseptic, ready-to-eat, ready-to-drink, or combinations thereof. In another embodiment, the composition can be used in a pharmaceutical product such as a medication, coating, solution, liquid, or combinations thereof.

In each of the above embodiments, the particles will generally exhibit altered or enhanced physical properties such as, for example, a high solubility rate, an apparent low bulk density, and/or improved adherence. Further, the particles will impart a similar taste, aroma and color to each portion of a food substrate to which the composition is applied. The particles together will form a composition that is a substantially uniform blend of the salt and the ingredient for use in a variety of products such as food, dietary, and pharmaceutical products.

### EXAMPLES

The uniformity of the particulates that make up the composition comprising a salt and an ingredient made by the present invention is evident from tests and analyses performed on the product and examination of the particulates in comparison with product mixed by typical processes. In the first example, tests are performed to compare the composition made from the present invention with products mixed together by methods known by those skilled in the art such as blending of dry ingredients together and plating liquids onto a salt such as sodium chloride. Samples of the composition of the present invention are prepared using the ingredients below prepared by the method disclosed herein. The samples consists of a control of an orange flavor powder (Cargill Flavor Systems orange flavor powder, #043-00300, 2% by weight) blended with a sodium chloride salt. The second sample consists of a composition prepared according to the present invention comprising salt and the orange flavor powder (Cargill Flavor Systems orange flavor powder, #043-00300, 2% by weight). The third sample consists of plating an orange flavor liquid (Cargill Flavor Systems orange flavor liquid, #040-00138, 0.2% by weight) onto a salt. The fourth samples consists of a composition prepared according to the present invention comprising salt and, as the ingredient, an orange flavor liquid (Cargill Flavor Systems orange flavor liquid, #040-00138, 0.2% by weight). A fifth sample is of a composition of sodium chloride and garlic oil (Vegetable Juices Inc. garlic oil, #10098, 0.2% by weight) prepared by the present invention.

Observations show that samples prepared by the present invention result in a composition that is a substantially uniform blend of the salt and ingredient. In contrast, product made from known methods results in a product where the salt is superficially coated with the ingredient. The samples are dropped on a slide and observed under a microscope using light from various angles. Microscopic evaluation of the composition and a typical particulate shows that it is a substantially uniform blend of the salt and ingredient throughout the composition and the ingredient including an orange colorant can be seen in the salt. In contrast, the microscopic evaluation of a traditional blended product shows the ingredient including the orange colorant primarily adhering to the surface of the salt. This is observed for both the composition and the traditional blended product whether they are in a water solution or an oil solution. In the traditional blended product, the ingredient is observed by color to wash away from the salt. In comparison, the composition of the present invention has more of the ingredient that can be seen in and throughout the salt. Furthermore, in the composition, both the salt and ingredient essentially dissolve together at the same time rather than as two separate compounds. In contrast, observations of a traditional blended product in a liquid show dissolution first of the ingredient followed by dissolution of the salt, indicating that the ingredient was only adhering to the surface of the salt. The benefits of the present invention include a reduction in the loss of a compound product through preparation, handling, transportation, and even consumption of the composition in comparison to traditional blended products where the ingredient can separate from the salt in any of these activities.

In the next set of tests summarized in TABLE 1 below, it can be seen how the samples of a composition comprising a salt and an ingredient where the particulates in the composition form a substantially uniform blended product of the two compounds exhibit several superior physical benefits over a standard blended product of salt and an ingredient. In particular, the tests run show that the bulk density of the disclosed composition is lower than that of a standard blended product, the composition has a greater solubility rate, and it has improved adherence to a substrate such as a food product than a standard blended product.

Samples were prepared in a salt pilot plant, including a Ludman 1206 Flake Mill. Except as noted otherwise in the table below, runs were made using untreated purified sea salt with 10% by weight roasted granulated garlic, at a roll pressure of either 1.0 or 1.33 tons per lineal inch, at a roll speed of 6.25 fps, at ambient temperature, with a product thickness of 0.008 inches, and without recycling back nonconforming material. Bulk density, friability, flowability, and adherence testing are done with particulates having an average particle size of about U.S. Standard Sieve size 20×30 screen fractions (USS 20×30) for products made by both methods. The standard blended product used for comparison comprised granulated salt blended with 10% by weight roasted granulated garlic, the resulting product having a thickness of 0.008 inches. For ease of review, any changes to the above parameters used for testing on the disclosed composition are grouped according to those changes in the table below. Specifically, runs with variable roll pressure, roll speed, recycling of product back into the process, feed salt temperature, feed salt particle size, flake thickness, seasoning level, and seasoning or flavoring type, are grouped together. All percentages herein are expressed in weight percentage.

TABLE 1

| | | | | | Bulk | Friability | | |
|---|---|---|---|---|---|---|---|---|
| | | Yield | | | Density | % | Flowability | Adherence |
| Run | | Extra Coarse | Coarse | Fine | lb/ft³ | Unbroken | g/sec | % |
| | Roll Pressure (tons | | | | | | | |
| | per lineal inch) | | | | | | | |
| 1 | 0.66 | 10% | 31% | 59% | 48.9 | 92.3 | 17.0 | |
| 3 | 0.99 | 20% | 35% | 45% | 51.5 | 94.9 | 17.5 | |
| 2 | 1.33 | 21% | 38% | 42% | 48.7 | 95.9 | 16.7 | |
| 4 | 2.7 | 17% | 38% | 46% | 45.3 | 95.1 | 14.7 | |
| | Roll Speed (fps) | | | | | | | |
| 3 | 6.25 | 20% | 35% | 45% | 51.5 | 94.9 | 17.5 | |
| 5 | 10.7 | 12% | 37% | 51% | 45.0 | 94.9 | 15.2 | |
| | Recycle | | | | | | | |
| 2 | 0 | 21% | 38% | 42% | 48.7 | 95.9 | 16.7 | |
| 6a | 50% | 22% | 30% | 48% | 47.7 | 92.7 | 16.3 | |
| 6b | 100% | 29% | 21% | 50% | 49.3 | 92.8 | 16.9 | |
| | Feed Salt Temperature | | | | | | | |
| 3 | Ambient | 20% | 35% | 45% | 51.5 | 94.9 | 17.5 | |
| 7 | Cold - 15° F. | 10% | 38% | 52% | 50.0 | 92.0 | 16.7 | |
| 8 | Hot - 120° F. | 13% | 36% | 51% | 50.0 | 92.7 | 17.6 | |
| | Feed Salt Particle Size | | | | | | | |
| 2 | Granulated | 21% | 38% | 42% | 48.7 | 95.9 | 16.7 | |
| 9 | Fine Blending | 13% | 31% | 56% | 47.4 | 91.8 | 14.8 | |
| | Flake Thickness | | | | | | | |
| 3 | 0.008 inches | 20% | 35% | 45% | 51.5 | 94.9 | 17.5 | 65 |
| 10 | 0.016 inches | 20% | 28% | 52% | 54.4 | 94.6 | 17.8 | 50 |
| 11 | 0.030 inches | 27% | 19% | 54% | 58.3 | 91.1 | 35.2 | 14 |
| | Seasoning Level | | | | | | | |
| 2 | 10% | 21% | 38% | 42% | 48.7 | 95.9 | 16.7 | |
| 12 | 15% | 19% | 35% | 45% | 51.0 | 94.3 | 15.8 | |
| 13 | 20% | 23% | 31% | 47% | 52.1 | 94.2 | 18.5 | |
| | Seasoning/Flavor | | | | | | | |
| | Type | | | | | | | |
| 2 | 10% Roasted | 21% | 38% | 42% | 48.7 | 95.9 | 16.7 | |
| | Granulated Garlic | | | | | | | |
| 14 | Dehydrated Garlic | 12% | 38% | 58% | 52.8 | 91.1 | 19.1 | |
| | Powder (a seasoning) | | | | | | | |
| 15 | Garlic Flavor, powdered, | 2% | 28% | 70% | 55.8 | 95.4 | 20.6 | |
| | #086-03181, Cargill | | | | | | | |

Standard Product

| | | | | |
|---|---|---|---|---|
| Granulated Salt, USS | 76.1 | 100 | 26 | 24.8 |
| 20 × 30, blended with | | | | |
| 10% Roasted | | | | |
| Granulated Garlic | | | | |

### Bulk Density

In all the tests run on the disclosed composition with the various conditions, the apparent bulk density of the disclosed composition is less than that for the standard product of salt blended with an ingredient. Under the same conditions (comparing Run #3 and the standard product in the final run), the bulk density for the standard blended product is 76.1 pounds per cubic foot (lb/cu ft) in comparison to the bulk density of 51.5 lb/cu ft for the disclosed composition, where both are blended with 10% by weight roasted granulated garlic and each has a flake thickness of 0.008 inches with a USS 20×30 screen fraction.

Products with lower bulk density can have a more irregular shape (e.g., Alberger® salt), a more porous structure, or both. Bulk density is typically expressed in weight per volume and a lower bulk density has the benefit of needing less material to have the same sensory experience. This can lead to the use of less material while maintaining the same benefits. By way of example, if sodium chloride is used as the salt, less sodium chloride can be used while still experiencing the same flavor profile of regular salt.

### Adherence

Adherence of the samples is measure by placing one gram of the product to be tested onto an Octogon 200 sieve shaker and distributing the particles by shaking them onto a stationary inclined plate uniformly coated with a thin film of vegetable oil. The angle of the plate is fixed at a standard position. The weight of the particles that adhere is compared to the one gram, and the percentage of the product adhering is calculated. It is generally done in replicate. Comparing the adherence of the standard blended product with the disclosed composition where both are blended with 10% by weight roasted granulated garlic and each has a flake thickness of 0.008 inches with USS 20×30 screen fraction (comparing Run #3 with the standard product), it can be seen that the composition has an adherence of 65% by weight to 24.8% by weight for the standard product. Even when using a higher flake thickness of 0.016 inches as seen in run #10, the adherence of the composition is 50% by weight compared to 24.8% by weight for the standard product. This greater adherence has the benefit especially for topical applications of the composition. The greater the adherence to the substrate, the less material is lost when applying it on. This will also lead to more of a flavor impact from the composition when it is consumed. Examples of substrates that benefit from greater adherence of the particulates include topical applications to products such as snacks, chips, popcorn, pretzels, meats, grains, and other food products, whether applied by a manufacture on a prepared or pre-packaged product or by the end-user.

### Solubility

The solubility is typically expressed as the time for 1,000 grams of salt, mixed in 3,000 ml of 60° F. water, to reach 90% saturation. The product is added to a standard container, fitted with a standard agitator, run at a standard speed. The water is added first, then the agitator is started, and then at time zero, the product is added. A Dicromat salinity tester is used to measure the time elapsed to reach 90% saturation. Calculations were performed because garlic interferes with the standard solubility test, however, these can be made fairly accurately based upon known solubility values. Using the information obtained on the tests above, calculations were made as to the solubility of the standard blended product in comparison to the disclosed composition where both are blended with 10% by weight roasted granulated garlic and each has a flake thickness of 0.008 inches with a USS 20×30 screen fraction (comparing Run #3 with the standard product). For a standard blended product the solubility time would be 42 seconds, in comparison to the solubility rate for the disclosed composition of 31 seconds. Faster solubility rates are beneficial in many applications. For example, when applying the composition onto a substrate, it is often applied in solution and it is preferable to have a compound with a faster solubility rate. This is also beneficial for the taste experience. The faster the salt goes into solution when consumed, the quicker the flavor is perceived. It is well-known that a faster solubility rate provides a “flavor burst” that brings out other flavors associated with the product. This is particularly important in the present application when the salt is combined with other ingredients. A product with a higher solubility rate will therefore result in a “flavor burst” in the mouth when consumed of not only the salt but the ingredient as well.

### Recycling Product

In some of the experiments, one of the variables was recycling material that had been separated out back into the process. Specifically, material that was oversized or undersized compared to the target product is recycled back into the process in the mixing, pressing or reducing step. In two runs 6a and 6b, 50% by weight and 100% by weight of these materials respectively were recycled back into the process resulting in a composition that is comparable to runs where no product is recycled.

### Operating Conditions

The above tests not only show the unique physical attributes of the claimed composition, but also are illustrative of the wide range of operating conditions in which the disclosed process will work. The tests are run by varying operating conditions in the pressing step, reducing step, mixing step, and separating steps by preparing the composition under various conditions of roll pressure, roll speed, recycle and feed temperature. In addition, compositions of varying particle size, flake thickness, seasoning level, and seasoning or flavoring type are used.

Further testing on the product in samples is performed on a variety of substrates, including a protein such as chicken, a carbohydrate such as cooked rice, and a snack item such as potato chips.

### Protein (Chicken) Application

In Table 2 below, testing is performed to determine the optimal blends of the salt and ingredient. In order to control the substrate, chicken patties are made from ground chicken to deliver a consistent thickness to the chicken. A fine blending salt is used with the roasted dehydrated granulated garlic as the bulk densities between them are similar and provide the most homogenous blend. After the chicken patties are prepared as described in the Procedure below, the disclosed composition is prepared according to the formulas in Table 2 below and added to the chicken patties, followed by sensory testing and evaluation. All six formulas are pre-screened by a smaller group of testers, and the preferred formulas are 2, 3, and 4. Mixtures are prepared using both the traditional blended product and the composition from the present invention for comparison and testing by tasters. A larger group tastes those formulas made by both traditional blending and the present invention for a final recommendation. Formula 3 is the more preferred composition prepared by the present invention for this application, although formula 2 is also deemed acceptable. Applied formulas are expressed in grams.

TABLE 2

| | | Control | 1 | 2 | 3 | 4 | 5 |
|---|---|---|---|---|---|---|---|
| | | Preliminary Formula | | | | | |
| Chopped, | | 99.20% | 99.20% | 99.20% | 99.20% | 99.20% | 99.20% |
| Cooked | | | | | | | |
| Chicken | | | | | | | |
| Patty | | | | | | | |
| Fine | Cargill | 0.80% | 0.80% | 0.80% | 0.80% | 0.80% | 0.80% |
| Blending | | | | | | | |
| Salt | | | | | | | |
| Roasted | Spice | 0.00% | 0.05% | 0.10% | 0.20% | 0.30% | 0.40% |
| Granulated | Barn | | | | | | |
| Garlic | | | | | | | |
| (dehydrated) | | | | | | | |
| | | 100.00% | 100.05% | 100.10% | 100.20% | 100.30% | 100.40% |
| | | Applied Formula (in grams) | | | | | |
| Chopped, | | 120.0000 | 120.0000 | 120.0000 | 120.0000 | 120.0000 | 120.0000 |
| Cooked | | | | | | | |
| Chicken | | | | | | | |
| Patty | | | | | | | |
| Fine | Cargill | 0.9677 | 0.9677 | 0.9677 | 0.9677 | 0.9677 | 0.9677 |
| Blending | | | | | | | |
| Salt | | | | | | | |
| Roasted | Spice | 0.0000 | 0.0605 | 0.1210 | 0.2419 | 0.3629 | 0.4839 |
| Granulated | Barn | | | | | | |
| Garlic | | | | | | | |
| (dehydrated) | | | | | | | |
| | | 120.9677 | 121.0282 | 121.0887 | 121.2096 | 121.3306 | 121.4516 |
| | | NORMALIZED TO 100% | | | | | |
| Chopped, | | 99.2000% | 99.1504% | 99.1009% | 99.0021% | 98.9033% | 98.8048% |
| Cooked | | | | | | | |
| Chicken | | | | | | | |
| Patty | | | | | | | |
| Fine | Cargill | 0.8000% | 0.7996% | 0.7992% | 0.7984% | 0.7976% | 0.7968% |
| Blending | | | | | | | |
| Salt | | | | | | | |
| Roasted | Spice | 0.0000% | 0.0500% | 0.0999% | 0.1996% | 0.2991% | 0.3984% |
| Granulated | Barn | | | | | | |
| Garlic | | | | | | | |
| (dehydrated) | | | | | | | |
| | | 100.0000% | 100.0000% | 100.0000% | 100.0000% | 100.0000% | 100.0000% |
| | | % Roasted Granulated Garlic in Garlic/Salt Blend | | | | | |
| Fine | Cargill | | 94.1% | 88.9% | 80.0% | 72.7% | 66.7% |
| Blending | | | | | | | |
| Salt | | | | | | | |
| Roasted | Spice | | 5.9% | 11.1% | 20.0% | 27.3% | 33.3% |
| Granulated | Barn | | | | | | |
| Garlic | | | | | | | |
| (dehydrated) | | | | | | | |
| | | | 100.0% | 100.0% | 100.0% | 100.0% | 100.0% |

Procedure
Form 150 gram ground, raw chicken (Perdue retail) into round patties measuring 4.25″ dia. × 0.5″ deep
Place patties onto parchment paper in a baking pan and cover with aluminum foil
Bake patties in 350 degree F oven for 20-25 minutes. Remove from oven, let rest 5 minutes, topically add the appropriate amount of salt and garlic per formulation above to each patty
Cook yield = 80%
Taste and evaluate

For the tasting and evaluation of the chicken products, a small group of trained taste testers and evaluators compared the products. The testers found that the chicken product using the disclosed composition has a higher impact of garlic than the blended product, with some roasted garlic notes. The blended product lacks the impact of the disclosed composition, and the profile is more blended and rounded out. This is consistent with the unique physical properties of the disclosed composition, including having a higher solubility rate and lower bulk density, as compared to a standard blended product.

Further testing is done with a small group evaluation consisting of 57 panelists to determine if there are perceived differences between the control blended product versus the composition made by the present invention both being applied at 1.0% by weight to the ground chicken patties. The methodology involves having the panelists evaluate the samples under red light in individual tasting booths, with the chicken patties served warm in odorless, tasteless plastic cups in a randomized order coded with random, three digit numbers. Fresh batches of the chicken are prepared and any remaining samples are thrown away rather than served. Panelists are served three samples, two that are the same and one that is different, and then are asked to evaluate the samples from left to right and choose the odd sample. Significance is determined at p<0.05 level using tables by Roessler, et al, 1978, Journal of Food Science 43:940-947.

The results show a statistically significant difference, with 26 out of 57 panelists evaluating correctly choosing the odd sample. The samples are significantly different at a value of p=0.036. Of the 26 panelists who correctly identify the odd samples, 21 indicated that the difference is greatest in the taste of the samples.

### Grain (Cooked White Rice) Application

Tests are also performed using the composition on cooked rice as seen on Table 3 below. In this set of tests, various compositions of a salt and an ingredient comprising garlic are mixed to determine the appropriate blend when applied topically to the cooked rice. In this instance a 10% by weight encapsulated garlic oils is used. However, due to the high strength nature of the encapsulated garlic oil, it is necessary to make a 10% by weight dilution by diluting it on the fine blending salt to aide in weighing accuracy. All six formulas are pre-screened by a smaller group of testers, and the preferred formulas are 4, 5, and 6. A larger group tastes those formulas for a final recommendation. Formula 5 is the more preferred composition for this rice application. Applied formulas are expressed in grams.

TABLE 3

| | | Control | 1 | 2 | 3 | 4 | 5 | 6 |
|---|---|---|---|---|---|---|---|---|
| | | Preliminary Formula | | | | | | |
| Cooked | Retail | 99.20% | 99.20% | 99.20% | 99.20% | 99.20% | 99.20% | 99.20% |
| Uncle Bens | | | | | | | | |
| Converted | | | | | | | | |
| Rice | | | | | | | | |
| Fine Blending | Cargill | 0.80% | 0.80% | 0.80% | 0.80% | 0.80% | 0.80% | 0.80% |
| Salt | | | | | | | | |
| 10% | Spice | 0.00% | 0.1000% | 0.2000% | 0.3000% | 0.0500% | 0.0250% | 0.0125% |
| Encapsulated | Barn | | | | | | | |
| Garlic Oil on | | | | | | | | |
| Salt | | | | | | | | |
| | | 100.00% | 100.1000% | 100.2000% | 100.3000% | 100.0500% | 100.0250% | 100.0125% |
| | | Applied Formula (in grams) | | | | | | |
| Cooked | Retail | 200.0000 | 200.0000 | 200.0000 | 200.0000 | 200.0000 | 400.0000 | 800.0000 |
| Uncle Bens | | | | | | | | |
| Converted | | | | | | | | |
| Rice | | | | | | | | |
| Fine Blending | Cargill | 1.6129 | 1.6129 | 1.6129 | 1.6129 | 1.6129 | 3.2258 | 6.4516 |
| Salt | | | | | | | | |
| 10% | Spice | 0.0000 | 0.2016 | 0.4032 | 0.6048 | 0.1008 | 0.1008 | 0.1008 |
| Encapsulated | Barn | | | | | | | |
| Garlic Oil on | | | | | | | | |
| Salt | | | | | | | | |
| | | 201.6129 | 201.8145 | 202.0161 | 202.2177 | 201.7137 | 403.3266 | 806.5524 |
| | | NORMALIZED TO 100% | | | | | | |
| Cooked | Retail | 99.2000% | 99.1009% | 99.0020% | 98.9033% | 99.1504% | 99.1752% | 99.1876% |
| Uncle Bens | | | | | | | | |
| Converted | | | | | | | | |
| Rice | | | | | | | | |
| Fine Blending | Cargill | 0.8000% | 0.7992% | 0.7984% | 0.7976% | 0.7996% | 0.7998% | 0.7999% |
| Slat | | | | | | | | |
| 10% | Spice | 0.0000% | 0.0999% | 0.1996% | 0.2991% | 0.0500% | 0.0250% | 0.0125% |
| Encapsulated | Barn | | | | | | | |
| Garlic Oil on | | | | | | | | |
| Salt | | | | | | | | |
| | | 100.0000% | 100.0000% | 100.0000% | 100.0000% | 100.0000% | 100.0000% | 100.0000% |
| | | % Roasted Granulated Garlic in Garlic/Salt Blend | | | | | | |
| Fine Blending | Cargill | | 88.8895% | 80.0010% | 72.7285% | 94.1180% | 96.9699% | 98.4616% |
| Salt | | | | | | | | |
| Roasted | Spice | | 11.1105% | 19.9990% | 27.2715% | 5.8820% | 3.0301% | 1.5384% |
| Granulated | Barn | | | | | | | |
| Garlic | | | | | | | | |
| (dehydrated) | | | | | | | | |
| | | | 100.0000% | 100.0000% | 100.0000% | 100.0000% | 100.0000% | 100.0000% |

Procedure
Stove top cooking: Combine rice and water (2 parts water: 1 part rice) in sauce pan, bring to boil and simmer covered for 20 minutes
Remove from heat. Let stand covered for 5 minutes until water absorbed. Fluff with fork.
Weigh appropriate amount of cooked rice onto plate, topically apply salt & garlic and stir into mixture and taste
Note:
Due to the high strength nature of the encapsulated garlic oil, it is necessary to make a 10% dilution by diluting it with the fine blending salt to aid in weighing accuracy.

The cooked rice products prepared above are also evaluated by a small group of trained taste testers to determine whether sensory testing can detect differences between the mixtures. The testers can detect a difference between them, with the disclosed composition having a lower flavor and aroma impact with cooked rice compared to the blended product, but having a garlic profile of fresh garlic. The blended product has less of a salt impact, but more impact with sulfury, dehydrated, and stale garlic notes. Overall, distinct differences are detected between the composition made from the disclosed process and the standard blended product.

Further sensory tests are run to determine if there are perceived differences between the control standard blended garlic salt versus the disclosed composition with each being applied at 0.825% by weight to Uncle Ben's converted white rice. The methodology is the same as used for the chicken patty as described above.

Once again panelists find a significant difference (p=0.009) between the samples, with 29 out of 59 panelists correctly choosing the odd sample. Of the 29 panelists who correctly choose the odd sample, 25 indicate the difference is greatest in the taste of the samples.

### Topical (Potato Chip) Application

### Shelf Life

In the final set of applications, tests are run on potato chips to determine the affect of shelf life between a standard blended product and the composition prepared by the present invention. Sensory tests are run to determine if there are perceived differences between the control standard blended garlic salt versus the composition with each being applied at 1.79% by weight to unsalted UTZ brand potato chips. Garlic powder from Spice Barn and fine blended sea salt from Cargill are used as the ingredient and salt with a final 10.1% by weight percent garlic powder level for both. The methodology is the same as used for the chicken patty as described above except the chips are served at room temperature and prepared, frozen and shipped to the testing location then stored at room temperature two weeks prior to testing. The samples are only opened as needed and only large pieces or whole chips are used in the evaluations. Panelists are instructed to eat an entire potato chip or two chips at once to get the best flavor evaluation.

In these aged samples, a significant difference (p=0.048) between the samples is found, with 22 out of 48 panelists correctly choosing the odd sample. All of the 22 panelists who correctly identified the odd sample indicate the difference is greatest in the taste of the samples. Comments indicate that the composition is perceived as stronger in garlic than the standard blended product. Also, it is noted that garlic is a very intense flavor for chip applications, and sensory fatigue or saturation can occur quickly. Nevertheless, the test results are consistent for all the products in finding a statistically significant sensory difference between a standard blended product and the composition made by the present invention indicating improved shelf life benefits.

### Solution Testing

Taste tests are also run to determine differences between product made from standard blends and from the disclosed composition in solution. One set of tests compares a standard dry blended product with product made from the disclosed composition, both made from a fine blending salt and a garlic powder with concentrations of 10.1% by weight garlic. They were evaluated in water at both 0.5% by weight salt and 1.2% by weight salt. The tests involve sampling and evaluation by four trained taste testers.

For the both salt solutions, consensus is reached that the aroma of the composition is stronger than the standard dry blend product. In addition, there is consensus that there is a difference in flavor profile between the two samples. Descriptors of the composition product include raw or green garlic, and some describe it as having an immediate strong garlic hit more than the standard dry blended product. One taster describes the composition product as flat, characteristic of reduced top notes. Two tasters describe the standard dry blended product as sweeter. Finally, there is no consensus on the 1.2% solution regarding overall intensity as it is too high to distinguish differences between the two products. Overall, definitive differences are found by taste between the disclosed composition and a standard dry blend product.

### Salts Testing

In the next set of tests, samples are taken comparing different types of salts such as flour salt (a very fine salt) with granulated salt. In these tests, as summarized in Table 4 below, samples are run using 0.2% by weight garlic oil as the ingredient combined with a granulated salt and with a flour salt. In one set the garlic oil is plated onto the salt as the standard process, and the other set is made using the present invention. In addition, different amounts of the ingredient garlic oil are combined with the salt to form the composition at 0.3% by weight garlic oil and 0.6% by weight garlic oil respectively.

As can be seen in Table 4 below, the amount of oil on the surface and total oil is consistent for the traditional plated blend of salt and garlic oil whether used with granulated salt or even with flour salt. The oil is on the surface of the salt, but it does not penetrate the salt and is not integrated into it. In comparison, the oil mixed with the salt, pressed together with it to form an agglomerate composition, and comminuted shows a high total oil content in relation to the amount of surface oil, indicating that the oil ingredient is entrapped or encapsulated into the salt, providing a substantially uniform blend of the salt and ingredient.

TABLE 4

| Samples | Surface Oil | Total Oil |
|---|---|---|
| 0.2% Garlic Oil plated on Granulated Salt | 0.14% | 0.14% |
| 0.2% Garlic Oil plated on Flour Salt | 0.15% | 0.15% |
| 0.2% Garlic Oil/Flour Salt Composition | 0.013% | 0.104% |
| 0.2% Garlic Oil/Granulated Salt Composition | 0.03% | 0.091% |
| 0.3% Garlic Oil/Granulated Salt Composition | 0.0042% | 0.0060% |
| 0.6% Garlic Oil/Granulated Salt Composition | 0.0046% | 0.0098% |

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

What is claimed:

1. A method of preparing a composition comprising the steps of:
(a) mixing a salt and an ingredient;
(b) pressing the salt and ingredient together to form an agglomerated composition; and
(c) comminuting the size of the agglomerated composition to a composition wherein the composition comprises particulates and a particulate comprises a substantially uniform blend of the salt and the ingredient.

2. The method of claim 1 wherein the pressing step is performed by compaction, flaking, pelletizing or briquetting.

3. The method of claim 1 wherein the agglomerated composition is formed into a sheet, flakes, pellets, or a briquette.

4. The method of claim 3 wherein the sheet is a continuous sheet having an average thickness of at least 0.005 inches.

5. The method of claim 4 wherein the sheet is a continuous sheet having an average thickness of about 0.005 inches to about 0.3 inches.

6. The method of claim 3 wherein the briquette has an average thickness of at least 0.25 inches.

7. The method of claim 6 wherein the briquette has an average thickness of about 0.25 inches to about 2.0 inches.

8. The method of claim 3 wherein the flakes have an average thickness of at least 0.004 inches.

9. The method of claim 8 wherein the flakes have an average thickness of about 0.004 inches to about 0.05 inches.

10. The method of claim 1 wherein the salt comprises an organic or inorganic salt comprising an anion of chloride, sulfate, acetate, carbonate, or gluconate or combinations thereof.

11. The method of claim 10 wherein the salt is sodium chloride, sea salt, potassium chloride, Fleur de Sel, iodized salt, or rock salt or combinations thereof.

12. The method of claim 1 wherein the ingredient comprises a flavoring agent, seasoning agent, coloring agent, aroma agent, masking agent, enhancing agent, high potency agent, compounded flavor, extract, nutrient, micronutrient, spice, oil, herb, mineral, inorganic salt, organic salt, salt substitute, sweetener, vitamin, emulsifying agent, stabilizing agent, anti-caking agent, dietary supplement, antioxidant or combinations thereof.

13. The method of claim 1 wherein the ingredient is a food ingredient generally recognized as safe for consumption.

14. The method of claim 12 wherein the coloring agent is selected from natural or artificial coloring agents.

15. The method of claim 1 further comprising the step of separating the composition according to a conforming size.

16. The method of claim 15 wherein at least a portion of a nonconforming composition is recycled back into the process in the mixing, pressing or reducing steps.

17. A composition comprising:
(a) a salt, and;
(b) an ingredient selected from the group consisting of a flavoring agent, seasoning agent, coloring agent, aroma agent, masking agent, enhancing agent, high potency agent, compounded flavor, extract, nutrient, micronutrient, spice, oil, herb, mineral, organic salt, inorganic salt, salt substitute, sweetener, vitamin, emulsifying agent, stabilizing agent, anti-caking agent, dietary supplement, and antioxidant, or combinations thereof, wherein the composition comprises particulates and a particulate comprises a substantially uniform blend of the salt and the ingredient.

18. The composition of claim 17 wherein the composition has a solubility rate faster than a corresponding product prepared by blending the salt and the ingredient having the same average particle weight.

19. The composition of claim 17 wherein the apparent bulk density of the composition is less than a corresponding product prepared by blending the salt and the ingredient having the same average particle weight.

20. The composition of claim 17 wherein the composition has an improved adherence to food than a corresponding product prepared by blending the salt and the ingredient having the same average particle weight.

21. The composition of claim 17 wherein the salt comprises an organic or inorganic salt comprising an anion of chloride, sulfate, acetate, carbonate, or gluconate or combinations thereof.

22. The composition of claim 21 wherein the salt is selected from the group consisting of sodium chloride, sea salt, potassium chloride, Fleur de Sel, iodized salt, and rock salt, or mixtures thereof.

23. The composition of claim 17 wherein the composition is used with a product, wherein the product comprises a food product, a dietary product, or a pharmaceutical product.

24. The composition of claim 23 wherein the food product comprises a meat, grain, vegetable, fruit, sauce, prepared meal, frozen food, candy, snack, chip, pre-packaged product, beverage, or combination thereof.

25. The composition of claim 23 wherein the dietary product comprises a dietary supplement, energy bar, beverage, powder, additive, energy drink, retort, aseptic, ready-to-eat, ready-to-drink, or combinations thereof.

26. The composition of claim 23 wherein the pharmaceutical product comprises a medication, coating, solution, liquid, or combinations thereof.
